(19) United States
(12) Patent Application Publication
(10) Pub. No.: US 2006/0005326 A1
(43) Pub. Date: Jan. 12, 2006

(54) Dyeing composition comprising at least one elastomeric film-forming polymer and at least one dyestuff

(76) Inventors: Isabelle Rollat-Corvol, Paris (FR); Jonathan Gawtrey, Boulogne (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/171,225

(22) Filed: Jul. 1, 2005

Related U.S. Application Data
(60) Provisional application No. 60/616,224, filed on Oct. 7, 2004.

(30) Foreign Application Priority Data
Jul. 1, 2004 (FR) 04 51390

(51) Int. Cl. A61K 8/00

(52) U.S. Cl. 8/405

(57) ABSTRACT

Disclosed is a dyeing composition comprising, in a medium suitable for dyeing, at least one dyestuff and at least one elastomeric film-forming polymer wherein the film obtained by drying the at least one elastomeric film-forming polymer at ambient temperature and at a relative humidity of 55% has properties comprising:
(a) an elongation at break (ε_{b}) of greater than or equal to 800%,
(b) an instantaneous recovery (Rᵢ) at least equal to 75%, after an elongation of 150%, and
(c) a recovery (R₃₀₀) at 300 seconds of greater than 80%.

This application claims benefit of U.S. Provisional Application No. 60/616,224, filed Oct. 7, 2004, the contents of which are incorporated herein by reference. This application also claims benefit of priority under 35 U.S.C. § 119 to French Patent Application No. 04 51390, filed Jul. 1, 2004, the contents of which are also incorporated by reference.

The invention relates to a dyeing composition comprising, in a medium suitable for dyeing keratin materials, at least one dyestuff and at least one film-forming polymer with specific elastomeric characteristics, and also to the method for dyeing keratin materials, for example, keratin fibers, and a process for applying a dyeing composition comprising, in a medium suitable for dyeing keratin materials, at least one dyestuff and at least one film-forming polymer with specific elastomeric characteristics.

For a long time, many individuals have been seeking to modify the color of their skin, of their eyelashes or of their hair, for example, to mask white hair. To do this, several technologies have been developed.

It is known practice to dye keratin fibers, for example, human hair, with dye compositions containing direct dyes. The conventional dyes that are used may be dyes of the nitrobenzene, anthraquinone, nitropyridine, azo, xanthene, acridine, azine or triarylmethane type, or natural dyes. These dyes may be non-ionic, anionic, cationic or amphoteric.

These dyes, which are colored or coloring molecules that have an affinity for the fibers, are applied to the keratin fibers for a period of time required to obtain the desired coloration, and then rinsed off.

The colorations that result may be particularly chromatic colorations which are, however, temporary or semi-permanent since the nature of the interactions that bind the direct dyes to the keratin fiber, and their desorption from the surface and/or from the core of the fiber, are responsible for their low dyeing power and their poor fastness with respect to washing or perspiration. These direct dyes are also, for example, sensitive to light, since the resistance of the chromophore with respect to photochemical attack is poor, which leads to fading of the coloration of the hair over time.

It is already known practice to introduce, into some direct dye compositions, polymers conventionally used for formulating hair products for styling. These polymers are, for example, anionic, amphoteric or non-ionic film-forming polymers that result in the formation of films having a more or less hard and brittle nature.

There is a need, in the dyeing field, for a composition that makes it possible to obtain at least one of strong colorations, in varied shades, that, over time, may withstand outside agents such as light, shampoo or sweat, while at the same time preserving the quality of the keratin material, for example, the ease of styling, especially satisfactory untangling, softness, and a pleasant, non-tacky appearance, when this composition is applied to the keratin fibers, and also ease of use. There is also a need for having dyeing compositions capable of forming on these keratin materials a flexible film that follows the movements of the skin or of the head of hair, without any effect of tautness or heaviness, or any rigid sensation.

An embodiment of this disclosure is a dyeing composition comprising, in a medium suitable for dyeing, at least one dyestuff and at least one elastomeric film-forming polymer wherein the film obtained by drying the at least one elastomeric film-forming polymer at ambient temperature and at a relative humidity of 55% has at least one property chosen from:
(a) an elongation at break (ε_{b}) of greater than or equal to 800%,
(b) an instantaneous recovery (Rᵢ) at least equal to 75%, after an elongation of 150%, and
(c) a recovery (R₃₀₀) at 300 seconds of greater than 80%.

In an embodiment, a composition disclosed herein may obtain strong colorations that withstand outside agents, while at the same time preserving the integrity of keratin materials and good styling, by means of the formation of a flexible, non-brittle film on human keratin materials, that follows their movements.

As used herein, the term “dyestuff” means any colored molecule that, when it is brought into contact with the keratin material, colors this material, or any non-colored molecule that, in contact with the keratin material, colors the keratin material without the aid of an additional chemical agent, for example, without the aid of an oxidizing agent. In one embodiment, the dyestuffs according to the disclosure are colored materials, for example colored molecules which, when they are in contact with the keratin material, color this keratin material.

Another subject of the present disclosure relates to a method for dyeing keratin materials, for example, keratin fibers, comprising applying at least one composition of the disclosure.

Another subject of the present disclosure relates to a process for dyeing keratin materials comprising applying at least one composition of the disclosure for dyeing keratin materials, for example, keratin fibers, such as the hair.

As used herein, the expression “film obtained by drying at ambient temperature and at a relative humidity of 55%” means the film obtained, under these conditions, from a mixture containing 6% of active material (a.m.) of elastomeric film-forming polymer in a mixture of 30% by weight of ethanol and 70% by weight of water, relative to the total alcohol+water weight, the amount of mixture being adjusted so as to obtain, in a teflon matrix, a film 500 μm±50 μm thick. The drying is continued until the weight of the film no longer changes, which represents approximately 12 days. The film-forming polymers that are soluble or partially soluble in ethanol are tested in ethanol alone. The other polymers are tested in water alone, in soluble or dispersed form.

For the purpose of the present disclosure, the elongation at break and the recovery rate are evaluated by means of the tests described below.

To carry out the tensile tests, the film is cut up into rectangular test pieces 80 mm long and 15 mm wide.

The tests are carried out on a device sold under the name Lloyd or sold under the name Zwick, under the same temperature and humidity conditions as for the drying, i.e. at ambient temperature and at a relative humidity of 55%.

The test pieces are drawn at a speed of 20 mm/min and the distance between the jaws is 50±1 mm.

To determine the instantaneous recovery (Rᵢ), the following procedure is carried out:
the test piece is drawn by 150% (εₘₐₓ), i.e. 1.5 times its initial length (l₀),
the stress is released by applying a return speed equal to the tensile speed, i.e. 20 mm/min, and the percentage elongation of the test piece is measured, after return to a zero constraint (εᵢ).

The % instantaneous recovery (Rᵢ) is given by the formula below:
Rᵢ=((εₘₐₓ−εᵢ)/εₘₐₓ)×100

To determine the recovery at 300 seconds, the test piece, having been subjected to the above operations, is maintained at zero stress for a further 300 seconds, and its percentage elongation (ε₃₀₀ₛ) is measured.

The % recovery at 300 seconds (R₃₀₀ₛ) is given by the formula below:
R₃₀₀ₛ=((εₘₐₓ−ε₃₀₀ₛ)/εₘₐₓ)×100

In an embodiment, the composition may optionally comprise at least one auxiliary film-forming agent, wherein the at least one elastomeric film-forming polymer and the an auxiliary film-forming agent form, under the conditions of the tests above, a film having an elongation at break ranging from 800% to 3000%; having an instantaneous recovery of 75% to 100%; and a recovery at 300 seconds ranging from 85% to 100%.

In another embodiment, the at least one elastomeric film-forming polymer is present in an amount ranging from 0.05% to 20% by weight, for example, from 0.1% to 15% by weight, and further, for example, from 0.25% to 10% by weight, relative to the total weight of the composition.

For example, the at least one elastomeric film-forming polymer may be chosen from polyurethanes, polyvinyl alcohols, and polymers comprising at least one (meth)acrylic unit. The at least one elastomeric film-forming polymer may be in the form of a homopolymer or of a copolymer. For example, the at least one elastomeric film-forming polymer is in a non-crosslinked form in the composition.

For example, the at least one elastomeric film-forming polymer may be soluble or hydrodispersible in an aqueous or aqueous-alcoholic medium. For example, the at least one elastomeric film-forming polymer may be soluble at at least 10 g of active material in 90 g of aqueous or aqueous-alcoholic medium (for example, comprising 70% water and 30% ethanol), at ambient temperature and atmospheric pressure.

According to another embodiment, the film formed from the composition of the disclosure has a low water sensitivity, for example in an atmosphere with a relative humidity of 30% to 80%, i.e. the film keeps its elastomeric properties for several hours. It is flexible and non-brittle and correctly follows the movements of the skin and/or of the head of hair. For example, ranging from 30% to 80% relative humidity, the elongation at break of the film obtained may not vary by more than 50% (±400%) and/or its instantaneous recovery does not vary by more than 25% (18.75%). In other words, ranging from 30% to 80% relative humidity, the film formed from the composition of the disclosure has at least one property chosen from an elongation at break of the film obtained ranging from 400% to 1200% and an instantaneous recovery ranging from 57% to 93%.

The elastomeric film-forming polymers that are useful in the composition of the disclosure are known from the art. They may, for example, be synthesized according to the method described in French Patent Application No. FR 2 815 350.

The at least one dyestuff may be chosen from direct, optionally fluorescent, dyes, pigments, DHA (dihydroxyacetone) and DHA derivatives.

The direct dyes are dyes that may be water-soluble or soluble in a solvent medium. The direct dyes may be chosen from neutral, acidic or cationic nitrobenzene direct dyes, neutral, acidic or cationic azo direct dyes, neutral, acidic or cationic quinone and, for example, anthraquinone direct dyes, azine direct dyes, triarylmethane direct dyes, indoamine direct dyes and natural direct dyes.

Among the benzene direct dyes, non-limiting mention may be made of the following compounds:
1,4-diamino-2-nitrobenzene
1-amino-2-nitro-4-β-hydroxyethylaminobenzene
1-amino-2-nitro-4-bis(β-hydroxyethyl)aminobenzene
1,4-bis(β-hydroxyethylamino)-2-nitrobenzene
1-1-hydroxyethylamino-2-nitro-4-bis(β-hydroxyethylamino)benzene
1-β-hydroxyethylamino-2-nitro-4-aminobenzene
1-β-hydroxyethylamino-2-nitro-4-(ethyl)(β-hydroxyethyl)aminobenzene
1-amino-3-methyl-4-β-hydroxyethylamino-6-nitrobenzene
1-amino-2-nitro-4-β-hydroxyethylamino-5-chlorobenzene
1,2-diamino-4-nitrobenzene
1-amino-2-β-hydroxyethylamino-5-nitrobenzene
1,2-bis(β-hydroxyethylamino)-4-nitrobenzene
1-amino-2-tris(hydroxymethyl)methylamino-5-nitrobenzene
1-hydroxy-2-amino-5-nitrobenzene
1-hydroxy-2-amino-4-nitrobenzene
1-hydroxy-3-nitro-4-aminobenzene
1-hydroxy-2-amino-4,6-dinitrobenzene
1-β-hydroxyethyloxy-2-β-hydroxyethylamino-5-nitrobenzene
1-methoxy-2-β-hydroxyethylamino-5-nitrobenzene
1-β-hydroxyethyloxy-3-methylamino-4-nitrobenzene
1-β,β-dihydroxypropyloxy-3-methylamino-4-nitrobenzene
1-β-hydroxyethylamino-4-β,β-dihydroxypropyloxy-2-nitrobenzene
1-β-dihydroxypropylamino-4-trifluoromethyl-2-nitrobenzene
1-β-hydroxyethylamino-4-trifluoromethyl-2-nitrobenzene
1-β-hydroxyethylamino-3-methyl-2-nitrobenzene
1-β-aminoethylamino-5-methoxy-2-nitrobenzene
1-hydroxy-2-chloro-6-ethylamino-4-nitrobenzene
1-hydroxy-2-chloro-6-amino-4-nitrobenzene
1-hydroxy-6-bis(β-hydroxyethyl)amino-3-nitrobenzene
1-β-hydroxyethylamino-2-nitrobenzene
1-hydroxy-4-β-hydroxyethylamino-3-nitrobenzene.

Among the azo direct dyes, non-limiting mention may be made of the cationic azo dyes described in PCT Patent Application Nos. WO 95/15144, WO 95/01772 and European Patent Application No. EP-714954, the content of which is incorporated herein by reference.

Non-limiting mention may be made of the following dyes:
1,3-dimethyl-2-[[4-(dimethylamino)phenyl]azo]-1H-imidazolium chloride,
1,3-dimethyl-2-[(4-aminophenyl]azo]-1H-imidazolium chloride, and
1-methyl-4-[(methylphenylhydrazono)methyl]pyridinium methyl sulphate.

Among the azo direct dyes, non-limiting mention may also be made of the following dyes, described in the Colour Index International, 3rd edition:
Disperse Red 17
Acid Yellow 9
Acid Black 1
Basic Red 22
Basic Red 76
Basic Yellow 57
Basic Brown 16
Acid Yellow 36
Acid Orange 7
Acid Red 33
Acid Red 35
Basic Brown 17
Acid Yellow 23
Acid Orange 24
Disperse Black 9.

Non-limiting mention may also be made of 1-(4′-aminodiphenylazo)-2-methyl-4-bis(β-hydroxyethyl)aminobenzene and 4-hydroxy-3-(2-methoxyphenylazo)-1-naphthalenesulphonic acid.

Among the quinone direct dyes, non-limiting mention may be made of the following dyes:
Disperse Red 15
Solvent Violet 13
Acid Violet 43
Disperse Violet 1
Disperse Violet 4
Disperse Blue 1
Disperse Violet 8
Disperse Blue 3
Disperse Red 11
Acid Blue 62
Disperse Blue 7
Basic Blue 22
Disperse Violet 15
Basic Blue 99
   and also the following compounds:
1-N-methylmorpholiniumpropylamino-4-hydroxyanthraquinone
1-aminopropylamino-4-methylaminoanthraquinone
1-aminopropylaminoanthraquinone
5-β-hydroxyethyl-1,4-diaminoanthraquinone
2-aminoethylaminoanthraquinone
1,4-bis(β,γ-dihydroxypropylamino)anthraquinone.

Among the azine dyes, non-limiting mention may be made of the following compounds:
Basic Blue 17
Basic Red 2.

Among the triarylmethane dyes, non-limiting mention may be made of the following compounds:
Basic Green 1
Acid Blue 9
Basic Violet 3
Basic Violet 14
Basic Blue 7
Acid Violet 49
Basic Blue 26
Acid Blue 7.

Among the indoamine dyes, non-limiting mention may be made of the following compounds:
2-β-hydroxyethylamino-5-[bis(β-4′-hydroxyethyl)amino]anilino-1,4-benzoquinone
2-β-hydroxyethylamino-5-(2′-methoxy-4′-amino)anilino-1,4-benzoquinone
3-N-(2′-chloro-4′-hydroxy)phenylacetamino-6-methoxy-1,4-benzoquinoneimine
3-N-(3′-chloro-4′-methylamino)phenylureido-6-methyl-1,4-benzoquinoneimine
3-[4′-N-(ethylcarbamylmethyl)amino]phenylureido-6-methyl-1,4-benzoquinoneimine.

Among the natural direct dyes that may be used according to the disclosure, non-limiting mention may be made of lawsone, juglone, alizarin, purpurin, carminic acid, kermesic acid, purpurogallin, protocatechaldehyde, indigo, isatin, curcumin, spinulosin and apigenidin. Extracts or decoctions comprising these natural dyes may also be used, for example, henna-based poultices or extracts.

The direct dyes that are useful in the present disclosure may also be fluorescent dyes. As used herein, the term “fluorescent dye” means a dye which is a molecule that colors by itself and therefore absorbs light in the visible spectrum and, optionally, in the ultraviolet spectrum (wavelengths ranging from 360 to 760 nanometres) but which, unlike a conventional dye, converts the energy absorbed into fluorescent light of higher wavelength, emitted in the visible part of the spectrum.

The fluorescent dyes that may be useful in the present disclosure may be differentiated from an optical brightening agent. Optical brightening agents, generally called brighteners, fluorescent brighteners, fluorescent brightening agents, fluorescent whitening agents, whiteners, or alternatively fluorescent whiteners, are colorless transparent compounds. These compounds do not color since they do not absorb in visible light, but only in the ultraviolet range (wavelengths ranging from 200 to 400 nanometers). They convert the energy absorbed into fluorescent light of higher wavelength, emitted in the visible part of the spectrum. The sensation of color is then only engendered by the purely fluorescent, predominantly blue light (wavelengths ranging from 400 to 500 nanometres).

The fluorescent dye used may be soluble in the medium of the composition or insoluble, like fluorescent pigments.

By way of examples of fluorescent dyes that may be used, non-limiting mention may be made of fluorescent dyes belonging to the following families: naphthalimides; cationic or non-cationic coumarins; xanthenodiquinolizines (such as sulphorhodamines); azaxanthenes; naphtholactams; azlactones; oxazines; thiazines; dioxazines; polycationic fluorescent dyes of the azo, azomethine or methine type, alone or as mixtures, for example, fluorescent dyes belonging to the following families: naphthalimides; cationic or non-cationic coumarins; azaxanthenes; naphtholactams; azlactones; oxazines; thiazines; dioxazines; polycationic fluorescent dyes of the azo, azomethine or methine type, alone or as mixtures.

Among the soluble fluorescent dyes, non-limiting mention may, for example, be made of:
Brilliant Yellow B6GL sold by the company Sandoz and having the following structure: 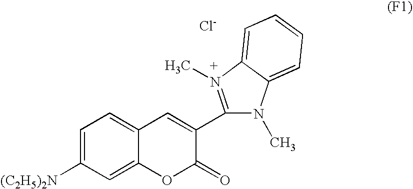
   Basic Yellow 2 or Auramine O, sold by the companies Prolabo, Aldrich or Carlo Erba and having the following structure:

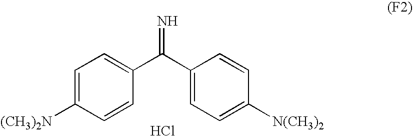

### 4,4′-(imidocarbonyl)bis(N,N-dimethylaniline) monohydrochloride —CAS number 2465-27-2

The composition of the disclosure may comprise pigments. In the context of the disclosure, the term “pigment” means any organic and/or mineral entity whose water-solubility is less than 0.01% at 20° C., for example, less than 0.001%, and which exhibits absorption ranging from 350 to 700 nm, for example, absorption with a maximum.

The pigments that are useful in the present disclosure may be chosen from all the organic and/or mineral pigments known from the art, for example, those which are described in the Encyclopaedia of Chemical Technology by Kirk-Othmer and in the Encyclopaedia of Industrial Chemistry by Ullmann or in “International Cosmetic Ingredient Dictionary and Handbook”, 1997 edition, pages 371 to 386 and 524 to 528, published by “The Cosmetic, Toiletry and Fragrance Association”.

These pigments may be, for example, in the form of a pigmentary paste or powder. They may be coated or uncoated.

The pigments in accordance with the disclosure may, for example, be chosen from white or colored pigments, lakes, and pigments with special effects such as pearlescent agents or flakes.

As examples of white or colored mineral pigments, non-limiting mention may be made of titanium dioxide, which may or may not be surface-treated, zirconium oxide or cerium oxide, iron oxide or chromium oxide, manganese violet, ultramarine blue, chromium hydrate and ferric blue.

As examples of white or colored organic pigments, non-limiting mention may be made of nitroso, nitro, azo, xanthene, quinoline, anthraquinone and phthalocyanin compounds, compounds of metallic complex type, and isoindolinone, isoindoline, quinacridone, perinone, perylene, diketopyrrolopyrrole, thioindigo, dioxazine, triphenylmethane and quinophthalone compounds.

For example, the white or colored organic pigments may be chosen from carmine, carbon black, aniline black, azo yellow, quinacridone, phthalocyanin blue, sorghum red, the blue pigments codified in the Colour Index under the references CI 42090, 69800, 69825, 73000, 74100, 74160, the yellow pigments codified in the Colour Index under the references CI 11680, 11710, 15985, 19140, 20040, 21100, 21108, 47000, 47005, the green pigments codified in the Colour Index under the references CI 61565, 61570, 74260, the orange pigments codified in the Colour Index under the references CI 11725, 15510, 45370, 71105, the red pigments codified in the Colour Index under the references CI 12085, 12120, 12370, 12420, 12490, 14700, 15525, 15580, 15620, 15630, 15800, 15850, 15865 15880 17200, 26100, 45380, 45410, 58000, 73360, 73915, 75470, the pigments obtained by oxidative polymerization of indole derivatives or phenolic derivatives as described in French Patent No. FR 2 679 771.

For example, pigmentary pastes of organic pigment may be used, such as the products sold by the company Hoechst under the name:
JAUNE COSMENYL IOG: Pigment YELLOW 3 (CI 11710);
   JAUNE COSMENYL G: Pigment YELLOW 1 (CI 11680);
   ORANGE COSMENYL GR: Pigment ORANGE 43 (CI 71105);
   ROUGE COSMENYL R: Pigment RED 4 (CI 12085);
   CARMIN COSMENYL FB: Pigment RED 5 (CI 12490);
   VIOLET COSMENYL RL: Pigment VIOLET 23 (CI 51319);
   BLEU COSMENYL A2R: Pigment BLUE 15.1 (CI 74160);
   VERT COSMENYL GG: Pigment GREEN 7 (CI 74260);
   NOIR COSMENYL R: Pigment BLACK 7 (CI 77266).

The pigments in accordance with the disclosure may also be in the form of composite pigments as described in European Patent No. EP 1 184 426. These composite pigments may be, for example, composed of particles comprising:
an inorganic core,
at least one binder for attaching the organic pigments to the core, and
at least one organic pigment that at least partially covers the core.

As used herein, the term “lake” means dyes adsorbed onto insoluble particles, the assembly thus obtained remaining insoluble during use. The inorganic substrates onto which the dyes are adsorbed are, for example, alumina, silica, calcium sodium borosilicate or calcium aluminium borosilicate, and aluminium. Among the organic dyes, non-limiting mention may be made of cochineal carmine.

As examples of lakes, non-limiting mention may be made of the products known under the following names: D & C Red 21 (CI 45 380), D & C Orange 5 (CI 45 370), D & C Red 27 (CI 45 410), D & C Orange 10 (CI 45 425), D & C Red 3 (CI 45 430), D & C Red 7 (CI 15 850:1), D & C Red 4 (CI 15.510), D & C Red 33 (CI 17 200), D & C Yellow 5 (CI 19 140), D & C Yellow 6 (CI 15 985), D & C Green (CI 61 570), D & C Yellow 10 (CI 77 002), D & C Green 3 (CI 42 053), D & C Blue 1 (CI 42 090).

As used herein, the term “pigments with special effects” means pigments that generally create a colored appearance (characterized by a certain shade, a certain vivacity and a certain level of luminescence) that is non-uniform and that changes according to the conditions of observation (light, temperature, angles of observation, etc.). They are consequently in contrast to white or colored pigments, which afford a standard opaque, semi-transparent or transparent uniform shade.

As examples of pigments with special effects, non-limiting mention may be made of white pearlescent pigments such as mica coated with titanium or with bismuth oxychloride, colored pearlescent pigments such as mica coated with titanium and with iron oxides, mica coated with titanium and, for example, with ferric blue or with chromium oxide, mica coated with titanium and with an organic pigment as defined above, and also pearlescent pigments based on bismuth oxychloride. Mention may also be made, by way of a pearlescent agent, of iridescent particles produced by certain molluscs in their shell.

Non-limiting mention may also be made of pigments with an interference effect that are not attached to a substrate, such as liquid crystals (Helicones HC from Wacker), holographic interference flakes (Geometric Pigments or Spectra f/x from Spectratek). The pigments with special effects also comprise fluorescent pigments, whether they are substances that are fluorescent in daylight or substances that produce ultraviolet fluorescence, phosphorescent pigments, photochromic pigments, thermochromic pigments and quantum dots, sold, for example, by the company Quantum Dots Corporation.

Quantum dots are luminescent semiconductive nanoparticles capable of emitting, under light excitation, radiation with a wavelength ranging from 400 nm to 700 nm. These nanoparticles are known in the literature. For example, they may be produced according to the processes described, for example, in United States Patent Nos. U.S. Pat. No. 6,225,198 or U.S. Pat. No. 5,990,479, and in the publications cited therein, and also in the following publications: Dabboussi B. O. et al. “(CdSe)ZnS core-shell quantum dots: synthesis and characterization of a size series of highly luminescent nanocrystallites” Journal of Physical Chemistry B, vol 101, 1997, pp. 9463-9475, and Peng, Xiaogang et al., “Epitaxial growth of highly luminescent CdSE/CdS core/shell nanocrystals with photostability and electronic accessibility” Journal of the American Chemical Society, vol 119, No. 30, pp. 7019-7029.

The variety of pigments that may be used in the present disclosure makes it possible to obtain a rich palette of colors, and also particular optical effects such as metallic or interference effects.

The size of a pigment other than the pearlescent agents in solution may range from 10 nm to 10 μm, for example, from 50 nm to 5 μm, and further, for example, from 100 nm to 3 μm. The size of a pearlescent agent in solution may range from 1 to 200 μm, for example, from 1 to 80 μm, and further, for example, from 1 to 50 μm.

The pigments may each be present in amounts ranging from 0.05% to 40% of the total weight of the composition, for example, from 0.1% to 35%.

As a dyestuff, non-limiting mention may also be made of dihydroxyacetone or its derivatives (subsequently referred to as DHA in the text). DHA has been known for a long time in the dyeing of keratin materials (Bobin et al. J. Soc. Cosmet. Chem., 35 pages 265-272,1984). DHA reacts with the amino acids naturally contained in the keratin material and, by virtue of a Maillard reaction, forms melanoids (Maillard L. C., C. R. Acad. Sci. 154, 66-68,1912). As compounds that are useful in the disclosure, non-limiting mention may be made of DHA and its derivatives as described in UK Patent Application No. GB 953 170, PCT Patent Application No. WO 96/09807 or French Patent Application No. FR-A-2597345.

The at least one dyestuff may be present in an amount ranging from 0.001% to 20% by weight, relative to the total weight of the ready-to-use composition, for example, from 0.005% to 10% by weight.

The composition of the present disclosure may also comprise oxidation bases and couplers conventionally used for oxidation dyeing.

By way of example, non-limiting mention may be made of para-phenylenediamines, bisphenylalkylenediamines, para-aminophenols, ortho-aminophenols, heterocyclic bases, and their addition salts.

The couplers may be, for example, chosen from meta-phenylenediamine couplers, meta-aminophenol couplers, meta-diphenol couplers, naphthalene couplers, heterocyclic couplers and their addition salts.

When they are present, the bases and the couplers are each present in an amount of ranging from 0.001% to 10% by weight, relative to the total weight of the dye composition, for example, from 0.005% to 6%.

The composition may also comprise at least one auxiliary film-forming agent such as a plasticizer and/or at least one agent for facilitating the formation of a film of the at least one elastomeric film-forming polymer on the keratin materials, the function of which is to modify the properties of the at least one elastomeric film-forming polymer. The at least one auxiliary film-forming agent may be chosen from all the compounds known to those skilled in the art as being capable of performing the desired function, and may, for example, be chosen from plasticizers and coalescence agents. The at least one elastomeric film-forming polymer, optionally combined with at least one auxiliary film-forming agent, is capable of forming a film, after evaporation of the cosmetic medium. This evaporation may be carried out in the open air or by providing heat, for example using a dryer.

The at least one auxiliary film-forming agent may be chosen from those described in French Patent No. FR-A-2 782 917. For example, this agent may be chosen from the usual plasticizers or coalescence agents, such as:
glycols and their derivatives, such as diethylene glycol ethyl ether, diethylene glycol methyl ether, diethylene glycol butyl ether, or else diethylene glycol hexyl ether, ethylene glycol ethyl ether, ethylene glycol butyl ether, ethylene glycol hexyl ether, pentylene glycol,
glyceryl esters,
propylene glycol derivatives, for example, propylene glycol phenyl ether, propylene glycol diacetate, dipropylene glycol butyl ether, tripropylene glycol butyl ether, propylene glycol methyl ether, dipropylene glycol ethyl ether, tripropylene glycol methyl ether, diethylene glycol methyl ether and propylene glycol butyl ether,
acid esters, for example, carboxylic acid esters, for example, citrates, phthalates, adipates, carbonates, tartrates, phosphates or sebacates, and
mixtures thereof.

The amount of the at least one auxiliary film-forming agent may be chosen by those skilled in the art based on their general knowledge, so as to obtain a polymeric system (elastomeric polymers+plasticizer and/or film-forming agent) that results in a film having the desired mechanical properties, while at the same time allowing the composition to conserve the desired cosmetic properties. For example, this amount ranges from 0.01% to 25% of the total weight of the composition, for example, from 0.01% to 15%.

The medium suitable for the dyeing operation, also called dye support, may consist of water or of a mixture of water and of at least one organic solvent so as to solubilize the compounds that are not sufficiently water-soluble. The at least one organic solvent, may be chosen from, for example, of C₁-C₄ lower alkanols, for example, ethanol and isopropanol; polyols and polyol ethers, for example, 2-butoxyethanol, propylene glycol, propylene glycol monomethyl ether, diethylene glycol monoethyl ether and monomethyl ether, and also aromatic alcohols such as benzyl alcohol or phenoxyethanol, and mixtures thereof.

For the dyeing of human keratin fibers, the dyeing medium is a suitable cosmetic medium.

The at least one organic solvent may be present in an amount ranging from 1% to 40% by weight, relative to the total weight of the dye composition, for example, from 5 to 30% by weight.

The dye composition in accordance with the disclosure may also comprise various adjuvants conventionally used in compositions for dyeing hair, such as anionic, cationic, non-ionic, amphoteric or zwitterionic surfactants or mixtures thereof, anionic, cationic, non-ionic, amphoteric or zwitterionic polymers or blends thereof, mineral or organic thickeners, for example, anionic, cationic, non-ionic and amphoteric polymeric, associative thickeners, antioxidants, penetrating agents, sequestering agents, fragrances, buffers, dispersants, conditioning agents such as, for example, volatile or non-volatile silicones, which may or may not be modified, film-forming agents other than those of the disclosure, for example, non-ionic, cationic, anionic or amphoteric fixing polymers, ceramides, preserving agents or opacifiers.

The above adjuvants may be present in an amount, for each of them, ranging from 0.01% to 20% by weight, relative to the weight of the composition.

Of course, those skilled in the art will take care to select this or these optional additional compound(s) in such a way that the advantageous properties intrinsically associated with the oxidation dyeing composition in accordance with the disclosure are not, or are not substantially, impaired by the addition(s) envisaged.

The pH of the dye composition in accordance with the disclosure may range from 3 to 12, for example, from 5 to 11.

The pH may be adjusted to the desired value by means of acidifying or basifying agents normally used in the dyeing of keratin fibers, or else by means of conventional buffer systems.

Among the acidifying agents, non-limiting mention may be made, of inorganic or organic acids such as hydrochloric acid, orthophosphoric acid, sulphuric acid, or carboxylic acids such as acetic acid, tartaric acid, citric acid or lactic acid, or sulphonic acids.

Among the basifying agents, non-limiting mention maybe made of aqueous ammonia, alkali metal carbonates, alkanolamines such as mono-, di- and triethanolamines and derivatives thereof, sodium hydroxide or potassium hydroxide, and the compounds of formula (III) below: 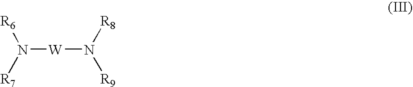
wherein:
W is chosen from a propylene residue optionally substituted with a group chosen from a hydroxyl group and C₁-C₄ alkyls; and R₆, R₇, R₈ and R₉, which may be identical or different, may be chosen from a hydrogen atom, C₁-C₄ alkyls and C₁-C₄ hydroxyalkyls.

The dye composition according to the disclosure may be in a form chosen from liquids, creams, gels, and in any other form that is suitable for dyeing keratin fibers, for example, human hair.

An embodiment of the disclosure is also a method of direct dyeing which comprises the application of the composition of the disclosure to keratin fibers and after a period of application, the keratin fibers are rinsed. The application time may range from 3 to 50 minutes, for example, from 5 to 30 minutes.

When the dye composition comprises an oxidation base and/or a coupler, the dye composition may also comprise at least one oxidizing agent. The oxidizing agents conventionally used for the oxidation dyeing of keratin fibers are, for example, hydrogen peroxide, urea peroxide, alkali metal bromates, persalts such as perborates and persulphates, peracids and oxidase enzymes among which non-limiting mention may be made of peroxidases, 2-electron oxidoreductases, for example, uricases and 4-electron oxygenases, for example, laccases. Hydrogen peroxide, for example, may be used.

The oxidizing agent may be added to the composition of the disclosure at the time of use, or it may be used from an oxidizing composition comprising it, applied simultaneously to or sequentially with the composition of the disclosure. The oxidizing composition may also comprise various adjuvants conventionally used in compositions for dyeing the hair and as defined above.

The pH of the oxidizing composition comprising the oxidizing agent is wherein, after mixing with the dye composition, the pH of the resulting composition applied to the keratin fibers ranges from 3 to 12, for example, from 5 to 11. It may be adjusted to the desired value by means of acidifying or basifying agents normally used in the dyeing of keratin fibers and as defined above.

In an embodiment, a composition of the disclosure that is applied to keratin fibers may be in a form chosen from liquids, creams, gels, and any other form suitable for dyeing keratin fibers, for example, human hair.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the embodiments disclosed herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosed embodiments are approximations, unless otherwise indicated the numerical values set forth in the specific examples are reported as precisely as possible. Any numerical value, however, inherently contain certain errors necessarily resulting from the standard deviation found in their respective testing measurements.

The embodiments disclosed herein are illustrated in greater detail by the examples described below.

### EXAMPLES

The amounts are given as percentages by mass, and A.M. signifies active material.

### Example 1

A dye composition was prepared, comprising:

| | |
|---|---|
| Polyurethane (NMDEA¹⁾/PTMO 2900²⁾/IPDI³⁾ − 3/1/4) | 2% AM |
| Basic Red 51 | 0.15 g |
| Ethanol | 5 g |
| 2-Amino-2-methyl-1-propanol | qs pH 8.5 |
| Demineralized water | qs 100 g |

¹⁾N-methyldiethanolamine
²⁾poly(tetramethylene oxide) having a weight-average mass of 2900
³⁾isophorone diisocyanate

The polymer was prepared according to the method of synthesis described in French Patent Application No. FR 2 815 350.

The polyurethane copolymer had the following characteristics, measured as above:
ε_{b}=1500%
Rᵢ=82%
R₃₀₀=92%
water-soluble at at least 10 g per liter.

This composition was applied to chestnut brown hair for 30 minutes and then rinsed out, conferring a persistent red effect after drying.

What is claimed is:

1. A dyeing composition comprising, in a medium suitable for dyeing, at least one dyestuff and at least one elastomeric film-forming polymer wherein the film obtained by drying the at least one elastomeric film-forming polymer at ambient temperature and at a relative humidity of 55% has properties comprising:
(a) an elongation at break (ε_{b}) of greater than or equal to 800%,
(b) an instantaneous recovery (Rᵢ) at least equal to 75%, after an elongation of 150%, and
(c) a recovery (R₃₀₀) at 300 seconds of greater than 80%.

2. A composition according to claim 1, wherein the at least one elastomeric film-forming polymer is soluble in an aqueous or aqueous-alcoholic medium.

3. A composition according to claim 1, wherein the at least one elastomeric film-forming polymer has properties, measured at a relative humidity ranging from 30% to 80%, chosen from an elongation at break of the film obtained ranging from 400% to 1200% and an instantaneous recovery ranging from 57% to 93%.

4. A composition according to claim 1, wherein the at least one elastomeric film-forming polymer is present in an amount ranging from 0.05% to 20% by weight.

5. A composition according to claim 1, wherein the at least one elastomeric film-forming polymer is present in an amount ranging from 0.1% to 15% by weight.

6. A composition according to claim 1, wherein the at least one elastomeric film-forming polymer is chosen from polyurethanes, polyvinyl alcohols, and polymers comprising at least one (meth)acrylic unit.

7. A composition according to claim 1, wherein the at least one dyestuff is chosen from direct dyes, fluorescent direct dyes, pigments, and DHA and its derivatives.

8. A composition according to claim 7, wherein the direct dyes are chosen from neutral, acidic or cationic nitrobenzene direct dyes, neutral, acidic or cationic azo direct dyes, neutral, acidic or cationic quinone direct dyes, azine direct dyes, triarylmethane direct dyes, indoamine direct dyes and natural direct dyes.

9. A composition according to claim 8, wherein the direct dyes are chosen from anthraquinone direct dyes.

10. A composition according to claim 7, wherein the direct dyes are fluorescent dyes.

11. A composition according to claim 7, wherein the pigments are chosen from white or colored pigments, lakes, and pigments with special effects.

12. A composition according to claim 1, wherein the amount of the at least one dyestuff is present in an amount ranging from 0.001% to 20% by weight, relative to the total weight of the composition.

13. A method for dyeing keratin materials, comprising applying to the keratin material at least on dyeing composition comprising, in a medium suitable for dyeing, at least one dyestuff and at least one elastomeric film-forming polymer wherein the film obtained by drying the at least one elastomeric film-forming polymer at ambient temperature and at a relative humidity of 55% has properties comprising:
(a) an elongation at break (ε_{b}) of greater than or equal to 800%,
(b) an instantaneous recovery (Rᵢ) at least equal to 75%, after an elongation of 150%, and
(c) a recovery (R₃₀₀) at 300 seconds of greater than 80%,
for a period of time sufficient to color the keratin materials.

14. A method according to claim 13, wherein the keratin materials are chosen from keratin fibers.

15. A process for dyeing keratin materials comprising applying to said fibers at least one dyeing composition comprising, in a medium suitable for dyeing, at least one dyestuff and at least one elastomeric film-forming polymer wherein the film obtained by drying the at least one elastomeric film-forming polymer at ambient temperature and at a relative humidity of 55% has properties comprising:
(a) an elongation at break (ε_{b}) of greater than or equal to 800%,
(b) an instantaneous recovery (Rᵢ) at least equal to 75%, after an elongation of 150%, and
(c) a recovery (R₃₀₀) at 300 seconds of greater than 80%.

16. A process according to claim 15, wherein the keratin materials are chosen from keratin fibers.
